(19) United States
(12) Patent Application Publication
(10) Pub. No.: US 2006/0004290 A1
(43) Pub. Date: Jan. 5, 2006

(54) Ultrasound transducer with additional sensors

(76) Inventors: Lowell Scott Smith, Niskayuna, NY (US); Rayette Ann Fisher, Niskayuna, NY (US); David Martin Mills, Niskayuna, NY (US); Charles Edward Baumgartner, Niskayuna, NY (US)

Correspondence Address: Patrick S. Yoder;FLETCHER YODER, P.O. Box 692289, Houston, TX, 77269-2289, US

(21) Appl. No.: 10/881,986

(22) Filed: Jun. 30, 2004

(51) Int. Cl. A61B 8/14

(52) U.S. Cl. 600/459

(57) ABSTRACT

The present technique provides for the manufacture and/or use of an ultrasound probe configured to acquire non-imaging data in addition to imaging data. In particular, the ultrasound probe includes a micro-machined ultrasound transducer formed on the surface of a substrate using micro-electric mechanical systems techniques or other techniques associated with semiconductor processing. Non-imaging sensors are formed on the substrate, either on the surface or the interior, or on a substrate proximate to the substrate upon which the transducer is formed. The non-imaging sensors may be used to acquire non-imaging data in conjunction with the acquisition of imaging data by the transducer.

### BACKGROUND

The invention relates generally to ultrasound imaging systems and more specifically to ultrasound probes for use in ultrasound imaging systems.

Various techniques have been developed which allow doctors and other medical personnel to generate images of the interior regions of a patient. One such technique is ultrasound imaging, which relies on the detection of sound waves to ascertain internal structure and composition of a patient. The data obtained from the detected sound waves may be processed to generate images or graphical representations, which may be reviewed and/or analyzed by a doctor or technologist to provide a diagnosis or other medical evaluation.

The interface between the ultrasound imaging system and the patient is an ultrasound transducer that is capable of converting electrical impulses and acoustic impulses, thereby enabling the generation and acquisition of the ultrasound data. In particular, the ultrasound transducer generates sonic waves, which propagate through the tissues of the patient, and measures acoustic reflections, which provide the information used to generate ultrasound images. The ultrasound transducer is typically incorporated in an ultrasound probe, which is typically a handheld unit that may be held and maneuvered by a medical technologist during the course of the examination of a patient. As will be appreciated by those of ordinary skill in the art, it may be desirable to acquire other data, such as temperature and/or position data, in conjunction with the ultrasound imaging process. In particular, such additional data may be useful in generating ultrasound images and/or in evaluating the imaging data.

Generally when additional data, such as temperature, position, and/or other data, is desired, separate discrete sensors are placed near the transducer, either on or separate from the ultrasound probe. For example, thermal monitoring may be performed by thermistors mounted in the transducer backing material or adjacent to the transducer of the ultrasound probe. Similarly, positioning data may be obtained using suitable positioning sensors, such as infrared or electromagnetic field position sensors, attached on or proximate to the ultrasound probe. Likewise, a switch hook or other proximity sensor mounted on the ultrasound probe may be used to provide data about patient contact or proximity, which may indicate or initiate a scanning operation. Similarly, other data of interest may be obtained using suitable sensors deployed near or on the probe.

However, such separate sensor arrangements may make the ultrasound imaging process more cumbersome and inconvenient for the operator. Therefore, it may be desirable to acquire other desired non-imaging data without the use of separate and/or distinct non-imaging sensors.

### BRIEF DESCRIPTION

According to one aspect of the present technique, an ultrasound probe is provided. The probe comprises an ultrasound transducer formed on a surface of a first substrate, a membrane and a cavity disposed between the surface of the first substrate and the membrane. The ultrasound probe further comprises one or more non-imaging sensors formed on the first substrate or on a second substrate proximate to the first substrate. In addition, an ultrasound imaging system comprising such an ultrasound probe is provided, as is a method of manufacturing such an ultrasound probe.

In accordance with another aspect of the present technique, a method for acquiring imaging and non-imaging data is provided. The method comprises contacting an ultrasound probe to a patient. The ultrasound probe comprises an ultrasound transducer formed on a surface of a first substrate and one or more non-imaging sensors formed on the first substrate or on a second substrate proximate to the first substrate. Ultrasound imaging data may be acquired via the ultrasound transducer. Non-imaging data may be acquired via the one or more non-imaging sensors.

### DRAWINGS

These and other features, aspects, and advantages of the present invention will become better understood when the following detailed description is read with reference to the accompanying drawings in which like characters represent like parts throughout the drawings, wherein:

FIG. 1 is a diagrammatic representation of an exemplary ultrasound system for use in conjunction with the present technique;

FIG. 2 is a cross-sectional top view of an exemplary ultrasound probe for use in conjunction with the present technique;

FIG. 3 is a perspective view of an ultrasound transducer substrate which includes sensors adjacent to a MUT array on the surface of the substrate, in accordance with one aspect of the present technique;

FIG. 4 is a perspective view of an ultrasound transducer substrate which includes sensors underlying a MUT array, in accordance with another aspect of the present technique;

FIG. 5 is a perspective view of an ultrasound transducer substrate which includes sensors underlying a MUT array, in accordance with a different aspect of the present technique;

FIG. 6 is a top view of a two-dimensional hexagonal array of ultrasound transducer sub-elements and square integrated position sensors, in accordance with one aspect of the present technique;

FIG. 7 is a diagrammatic illustration of embedded strain sensors in accordance with one aspect of the present technique;

FIG. 8 is a diagrammatic illustration of embedded strain sensors as illustrated in FIG. 7, where the development of localized strain is shown, in accordance with one aspect of the present technique; and

FIG. 9 is the diagrammatic illustration of an exemplary embedded bar code reader fabricated within an ultrasound transducer, in accordance with one aspect of the present technique.

### DETAILED DESCRIPTION

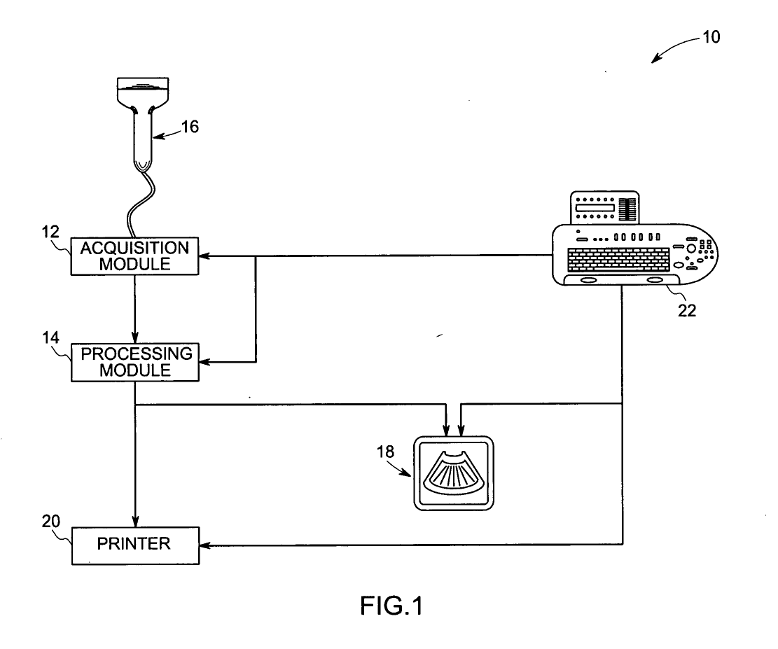

In the field of medical imaging, various imaging modalities may be used to non-invasively generate still or moving images of interior regions of a subject. For example, ultrasound imaging systems use the propagation and reflection of sonic waves to generate images representing the internal organs or structure of the subject. An example of such an ultrasound system is depicted in FIG. 1, which depicts an exemplary ultrasound system 10 for use in accordance with the present techniques. Though the example of a medical imaging implementation is discussed throughout, one of ordinary skill in the art will appreciate that other ultrasound imaging implementations may also benefit from the present techniques. For example, ultrasound imaging implementations may also be employed in the context of non-destructive evaluation (NDE) of materials, such as castings, forgings, or pipelines. It is to be understood that these and other ultrasound imaging embodiments may benefit from the present techniques and that the discussion of the present techniques in a medical ultrasound imaging context is merely provided as an example and is not intended to be limiting.

The exemplary ultrasound system 10 includes an acquisition module 12 and a processing module 14. Ultrasound system 10 generates ultrasonic signals, typically within a frequency range of 2 to 15 MHz, by means of an ultrasound probe 16 and transmits them into the body of the subject. In addition, the ultrasound probe 16 acquires reflected acoustic energy, which may be processed to generate graphical representations of the internal structures and/or composition of the body of the subject. As will be appreciated by those of ordinary skill in the art, different types of ultrasound probes may be employed with the exemplary ultrasound system 10, including linear, convex, micro-convex, sector, or intra-cavity probes.

The ultrasound probe 16 typically includes an ultrasound transducer array that converts electrical and acoustic energy close to the interface of the probe 16 and the body of the subject. In particular, a beamformer, which may be a component of the acquisition module 12, may generate the electrical signals that are converted to ultrasonic signals at the transducer and propagated in the body of the subject. Similarly, acoustic reflections, i.e., backscatter, may be converted at the transducer into electrical signals, which may then be transmitted from the probe 16 to the acquisition module 12 and, subsequently, to the processing module 14 for image reconstruction. Reconstructed images may be displayed on a display 18 or printed on a printer 20. As will be discussed in greater detail below, the ultrasound probe 16 may also acquire non-imaging data, such as temperature, pressure, strain, and/or optical data. The acquisition module 12 may control acquisition of non-imaging data. The acquired non-imaging data may be used by the acquisition module 12 in the acquisition of ultrasound imaging data. For example, strain or pressure indicators may indicate that the probe 16 is in acoustic contact with the subject. Similarly, an optical reader may provide optical data needed to identify the subject and, thereby, the record of the subject associated with the ultrasound imaging data. Alternatively, the acquired non-imaging data may be used by the processing module 14 to properly process the ultrasound imaging data. For example, temperature data may be a factor relevant to the reconstruction process performed at the processing module 14.

The operator or clinician may control the various processes of the exemplary ultrasound system 10, such as data acquisition, data processing, image display, and/or image printing, via an operator interface 22. As will be appreciated by those of ordinary skill in the art, a general purpose computer configured with suitable software, hardware, and/or peripherals may perform the functions of one or more of the acquisition module, the processing module, and/or the operator interface. Alternatively, a suitably configured special purpose platform, such as an application specific integrated circuit (ASIC), may perform the functions of one or more of the acquisition module, the processing module, and/or the operator interface. Therefore, in practice, the acquisition module 12, the processing module 14, and the operator interface 22 may reside on a single or on separate platforms, i.e., on one or more general purpose computers and/or ASICs.

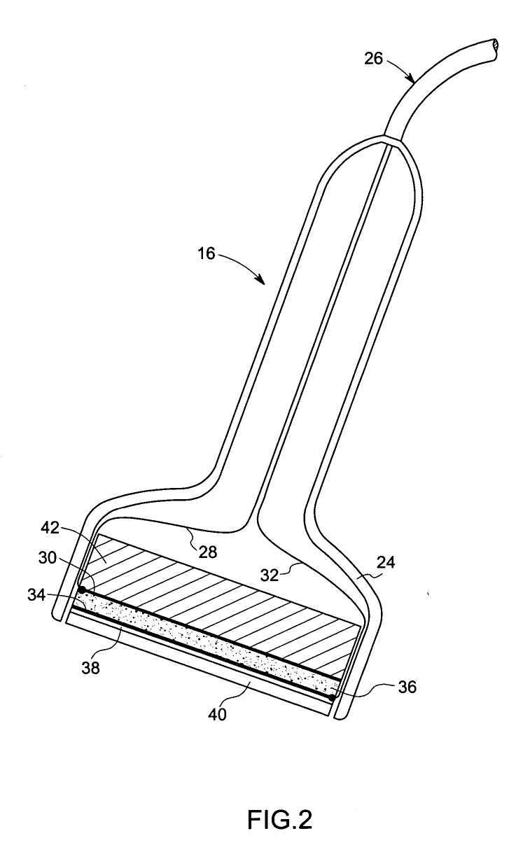

FIG. 2 is a cross-sectional top view of the exemplary ultrasound probe 16 of FIG. 1, for use in conjunction with the present technique. The ultrasound probe 16 comprises a housing 24, which is typically coupled to the acquisition module 12 by a cable assembly 26. The illustrated cable assembly 26 may be a coaxial cable or may include a plurality of miniature coaxial cables, such as between about 35 and about 1200 miniature coaxial cables. Though coaxial cables and bundles of miniature coaxial cables are possible implementations of the cable assembly 26, other suitable cables and/or conductive media may form the cable assembly 26.

A positive potential wire 28, which is connected to positive electrode 30, and a ground wire 32, which is connected to ground 34, provides electrical connectivity between the ultrasound probe 16 and the acquisition module 12. In particular, a transducer array 36 disposed between the positive electrode 30 and ground 34 may be electrically coupled to the acquisition module 12. The transducer array 36 may be a capacitive micro-machined ultrasound transducer (cMUT), as depicted in FIG. 2, or a piezoelectric micro-machined ultrasound transducer (pMUT).

As will be appreciated by those of ordinary skill in the art, a cMUT is composed of an array of transducer elements. Each element may include multiple capacitor cells. When a voltage is applied between a metalized membrane of the capacitive cells and the substrate upon which the cells are formed, the membrane vibrates, producing ultrasonic energy, which may be directed toward a subject. Likewise, during readout, the motion of a membrane in response to reflected acoustic energy may be detected as variations in electric charge or voltage, allowing the intensity of the reflection to be determined. An acoustic matching layer 38 and an acoustic lens 40 may also be present in the ultrasound probe 16 to protect the surface of the transducer array 36 and to focus the emitted ultrasound energy to a pre-selected focal depth into the body of the subject. A backing layer 42 may also be present in the ultrasound probe 16 to improve the acoustic response of the transducer and/or prevent external acoustic interference with the transducer array 36.

As will be appreciated by those of ordinary skill in the art, transducer arrays 36, such as cMUT or pMUT arrays may be formed on a substrate, such as a silicon substrate. Alternatively, the substrate may be formed from other materials, such as gallium arsenide, glass, or ceramic substrate, when suitable for an application. Though the substrate is discussed and depicted herein as a single, contiguous structure, the substrate may be a multi-layer structure, such as where different layers of the substrate include different circuits and/or structures. It may be noted that an acoustically lossy material may support the different layers of the substrates. For example, the substrate may be formed by a series of deposition and micro-machining processes, such as described below, such that different circuits, properties, structures and/or components are associated with each layer. Alternately, the multiple layers of such a multi-layer structure may be separate and discrete mediums, i.e. substrates, which are placed in contact or proximity during the manufacturing process.

The transducer array 36 and/or other circuitry may be formed on the substrate using micro-electric mechanical systems (MEMS) techniques or other semiconductor processing methods. By these fabrication techniques, micro-machined ultrasound transducer (MUT) layers of 0.5 to 5 microns thickness may be formed. In addition, and as discussed below, non-imaging sensors may also be fabricated on the same substrate either adjacent to, interspersed with, or beneath the elements of the transducer array 36.

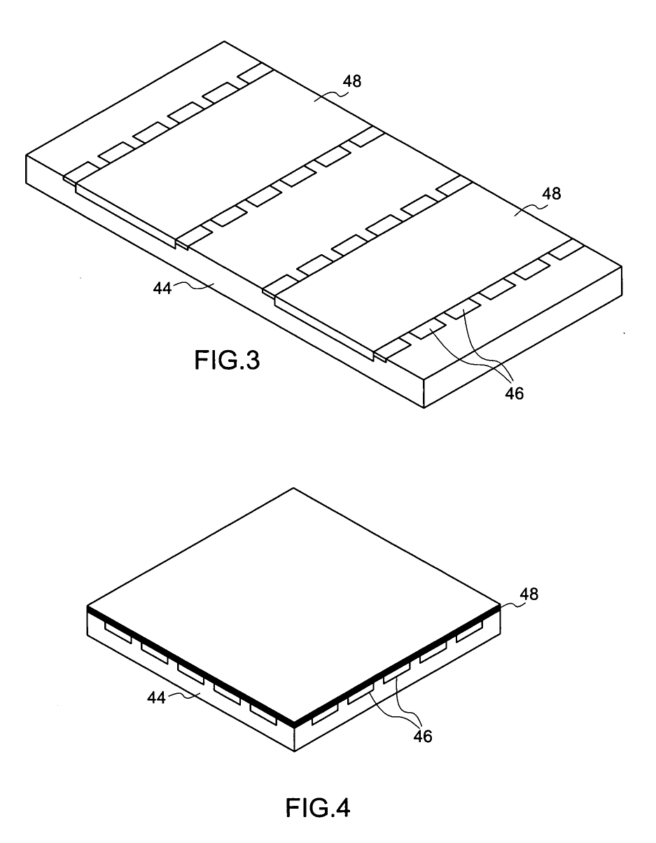

One such sensor arrangement is illustrated in FIG. 3. The illustration shows a perspective view of an ultrasound transducer substrate 44 upon which non-imaging sensors 46 have been formed adjacent to MUT arrays 48 on the surface of the substrate 44. The non-imaging sensors 46 may be fabricated on the substrate 44 using MEMS techniques or other semiconductor fabrication processes, such as surface micro-machining techniques or surface lithography. As depicted, the non-imaging sensors 46 may be situated along the edge or edges of the transducer array or arrays to form a linear or densely sampled array.

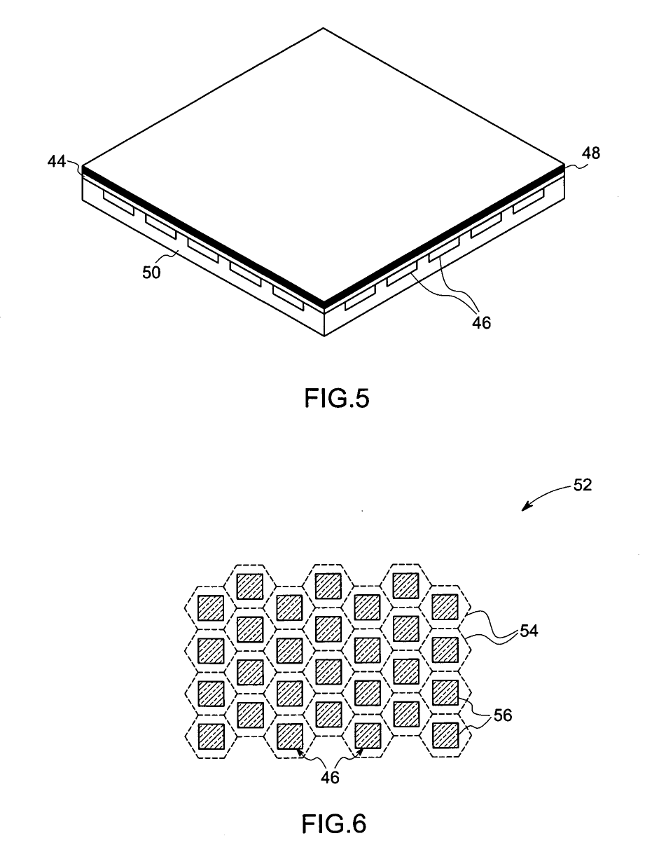

Similarly, FIG. 4 shows another arrangement for the non-imaging sensors 46. In this arrangement, the non-imaging sensors 46 are situated beneath the MUT array 48 on the substrate 44. As discussed above, MEMS techniques and/or other semiconductor fabrication techniques may be used to fabricate the underlying non-imaging sensors 46 and the overlying MUT array 48 on the substrate 44. For example, in a multi-layer substrate, the non-imaging sensors 46 may be fabricated on a layer of the substrate 44 directly or proximately underlying the layer upon which the MUT array 48 is formed. If desired, vias may electrically connect the MUT array 48 and non-imaging sensors 46 disposed on one or more underlying layers. Alternatively, as depicted in FIG. 5, the non-imaging sensors 46 may be formed on a separate substrate 50 directly or proximately underlying the substrate 44 upon which transducer array 48 is formed. In this manner, one or more non-imaging sensors 46 may acquire data concurrently with the MUT array 48 at a common location on the body of the subject. Similarly, non-imaging sensors may also overlie the transducer array if the non-imaging sensors are sufficiently acoustically transparent, such that the non-imaging sensors do not interfere with the transmission and reception of ultrasonic energy to and from the body of the subject.

The preceding discussion sets forth the fabrication and disposition of non-imaging sensors 46 on a common or proximate substrate upon which a transducer array 36, i.e., MUT array 48 is formed. Examples of specific implementations of non-imaging sensors 46 provided with a transducer array 36 will now be discussed to illustrate implementations of the present techniques. One example of a non-ultrasound imaging sensor 46, which may be provided with a transducer array 36 in this manner, is a thermistor.

In particular, temperature data from such a thermistor may be useful during the course of an ultrasound examination for regulating the operational state of the ultrasound probe 16. For example, to improve ultrasonic signal penetration, it may be desirable to operate the probe 16 near the maximum permitted temperature. It may therefore be desirable to fabricate thermal sensors or thermistors on the substrate surface or on an underlying layer of the substrate 44 or underlying substrate 50 to allow acquisition of thermal data in conjunction with ultrasound data. For example, such a thermistor may be fabricated by deposition of a resistive material and/or metallic alloy, such as nichrome. However, other resistive materials or temperature sensing elements that can be fabricated using MEMS or other semiconductor processing techniques may be used to achieve the same results. For example, thermistors can be fabricated using a bipolar p-n junction or multiple bipolar p-n junctions. In this way, one or more thermistors may be provided in conjunction with a MUT array 48 such that thermal data may be acquired at the site of contact with the subject, allowing regulation of the operation of the probe to be based on this thermal data.

One or more of the non-imaging sensors 46 may also be configured to acquire pressure data or other data indicative of contact of the probe 16 with the subject. For example, a pressure sensor may be placed proximate to a MUT array 48 to sense contact with the subject. Such a pressure sensor may be useful to indicate when too much pressure is being applied to the subject and/or probe 16 or to indicate when there is sufficient contact between patient and probe 16 to allow acquisition of ultrasound imaging data. For example, when the probe 16 contacts the subject with sufficient force to displace acoustic gel and, thereby, to achieve proper acoustic contact the pressure data provided by such pressure sensors may allow activation of the circuitry associated with acquisition of ultrasound imaging data or display of image data. Conversely, pressure data indicative of poor contact with the subject may be used to automatically inactivate the circuitry associated with acquisition of ultrasound imaging data, to provide notice to the operator of poor contact, such as via an indicator light or audible signal, and/or to inactivate the display of data. One example of a type of pressure sensor which may be used in this context and which may be fabricated on or under the substrate 44 by MEMS or other semiconductor processing techniques is a uniaxial pressure sensor. However, other types of pressure sensors, such as pressure sensors that operate on capacitive pressure sensing, piezoresistive pressure sensing, electromagnetic pressure sensing, or by other pressure sensing techniques may also be suitable.

In yet another implementation, it may be desirable for one or more of the non-imaging sensors 46 to acquire data concerning the position and/or orientation of the MUT array 48. In particular, such position and/or orientation data may be useful to facilitate the reconstruction of three-dimensional images using ultrasound imaging data since such three-dimensional reconstruction processes typically require spatial correlation of successive image frames. In addition, position and/or orientation information may also be used to derive information regarding deformation of the MUT array 48. In this manner, information concerning the deformation of the MUT array 48 may be used to implement compensatory processing of the image data or to generate a notification to the operator of the deformation or degree of deformation.

Non-imaging sensors capable of detecting position and/or orientation of the MUT array 48 can be constructed by MEMS techniques and/or other semiconductor processing techniques and may be situated adjacent to or underneath the MUT array 48, as described herein. For example, position and/or orientation sensors may be built with proof masses on cantilever beams and may provide information on position, orientation, and/or motion along different axes relative to the probe 16. One example of a type of non-imaging sensor that may be used to provide position and/or orientation information is uniaxial acceleration sensor. For example, a plurality of identical uniaxial acceleration sensors may be mounted in different orientations such that the position and/or orientation of the probe 16 may be determined from the aggregate output of the uniaxial acceleration sensors. Other types of position sensors may be employed, however, including inertial position sensors, electromagnetic position sensors, and optoelectronic position sensors, such as, infrared position sensors.

While the preceding discussion relates to MUT arrays 48 in general, those of ordinary skill in the art will appreciate that a MUT array 48 is typically a one or two-dimensional array 52 of MUT sub-elements 54, as depicted in FIG. 6. In such cases, it may be desirable to associate a non-imaging sensor 46, such as integrated position sensors 56, with some or all of the sub-elements 54. In the depicted implementation, an integrated position sensor 56 is located beneath each sub-element 54, however, integrated position sensors 56 may also be disposed under alternating sub-elements or according to some other pattern or array. Indeed, the arrangement of integrated position sensors 56 need not correspond directly to the array of sub-elements 54 so long as it is possible to associate position and/or orientation information from the integrated position sensors 56 with particular sub-elements 54. By having accurate position and/or orientation information for individual sub-elements 54, it may be possible to improve the quality of the ultrasound beam formed by the probe 16 during an examination.

In another implementation, one or more of the non-imaging sensors 46 may be a strain or displacement sensor for determining the deformation of the MUT array 48. While measurement of such deformation may be conducted using position and/or orientation sensors, as discussed above, it may instead be desirable to measure such deformation using such strain or displacement sensors. As will be appreciated by those of ordinary skill in the art, a variety of strain or displacement sensors may be suitable for fabrication on a substrate supporting a MUT array 48 or on an adjacent or proximate substrate. For example, sensors which measure strain or displacement based on capacitive changes, piezoresistive properties, or other electrical or physical indicators may be employed in accordance with the present techniques.

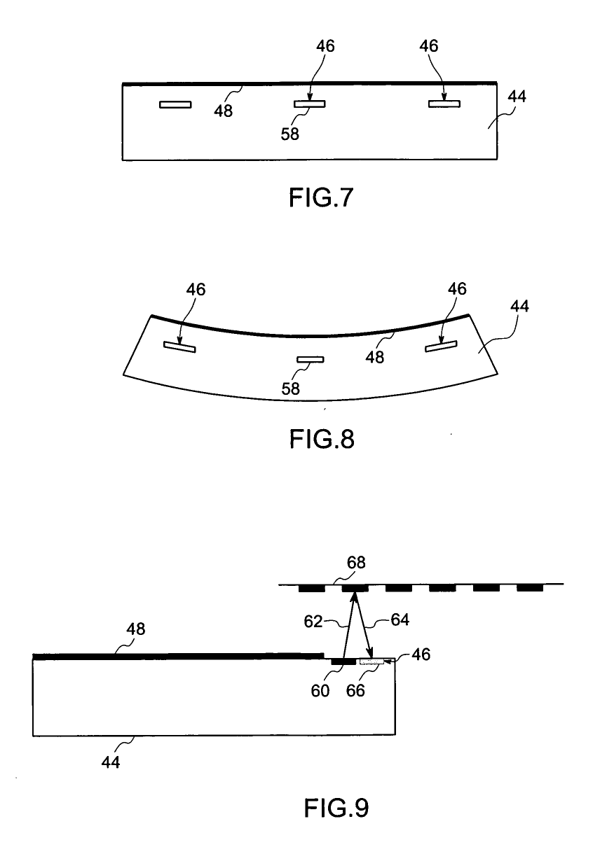

Referring to FIG. 7 and FIG. 8, diagrammatic view of a MUT array 48 formed on the surface of a substrate 44 is provided. Within the substrate 44 and beneath the MUT array 48, a series of strain sensors 58 are depicted. As noted above, the strain sensors 58 may be fabricated using MEMS techniques or other semiconductor fabrication techniques which allow different types of circuits to be produced at different levels within a substrate or on the surfaces of associated substrates. As depicted in FIG. 7, when the substrate 44 is not deformed, no localized strain is developed and the integrated strain sensors 58 are not deformed. However, as depicted in FIG. 8, when the substrate 44 bows, the integrated strain sensors 58 develop localized strain or displacements. Data from the strain gauge sensors 58 within the substrate 44 may thus indicate bending of the array under applied pressure, which may be used during processing to compensate for the distortion of the MUT array 48.

For example, bending of the substrate 44 can be estimated by a quadratic function using three to five strain sensors 58. The estimate of transducer deformation obtained in this manner may allow the determination of the relative positions of the MUT array elements. The number of strain sensors 58 employed may vary, however, depending on the extent or area of the MUT array 48, the amount of strain information desired, and the degree of precision desired with measurements. The deviations from a flat array surface may be used to modify the operation of the probe 16, such as by modifying the beamforming coefficients, to improve the resolution obtained.

In accordance with another implementation of the present techniques, the non-imaging sensors 46 may also be employed for patient identification or record access. For example, one or more of the non-imaging sensors 46 may include a reader, such as an optical or bar code reader or a radio-frequency identification (RFID) tag reader. Such a reader may be used to read a bar code, RFID tag, or other marker on or in a bracelet of the subject or on a medical chart associated with the subject.

For example, FIG. 9 depicts a bar code reader within the substrate 44, such as along a periphery of the MUT array 48. As shown, an optical source 60, and a photo-detector 66, may be formed on the surface of the substrate 44. The optical source 60 may be configured to emit light 62 while the photo-detector 66 may be configured to detect the reflected light 64. By moving the probe 16 along a bar code 68, data encoded by the bar code can be read. The optical source 60 may be any optoelectronic source, such as a light emitting diode, while the photo-detector 66 may be any optoelectronic receptor known in the art, such as photo-diodes.

In a related implementation, one or more non-imaging sensors 46 may be RFID readers which may be used to read an RFID tag containing patient records or identifying information. For example, a radio frequency interrogator or an electromagnetic sensor for reading radio frequency tags, may be fabricated either underneath or adjacent to the MUT array 48. In such an implementation, the reader can compare the information from the subject identification tag with the record of the subject in the healthcare facility database and associate it with the images and data obtained during the ultrasound examination. Thus, subject data can be updated and recorded in an automated manner. Similarly, a corresponding implementation may be utilized in non-destructive evaluation applications, such as where the subject being examined is a casting, forging, or other part. In such an implementation, a non-imaging sensor 46, such as an RFID reader, may facilitate the maintenance of a record of the findings and/or the association of the findings with the history of the analyzed parts.

While the preceding examples generally relate non-imaging sensors 46 that do not work on ultrasound principles, it should be understood that the non-imaging sensors 46 may also work on ultrasound principles. For example, the non-imaging sensors 46 may include ranging sensors that operate based on ultrasound techniques to detect proximity of the subject relative to the ultrasound probe. In such an embodiment, the ultrasound ranging sensor may provide information on proximity or contact which may be employed in the operation of the ultrasound probe 16 or the analysis of acquired data.

While only certain features of the invention have been illustrated and described herein, many modifications and changes will occur to those skilled in the art. It is, therefore, to be understood that the appended claims are intended to cover all such modifications and changes that fall within the true spirit of the invention.

1. An ultrasound probe, comprising:
an ultrasound transducer formed on a surface of a first substrate; and
one or more non-imaging sensors formed on the first substrate or on a second substrate proximate to the first substrate.

2. The ultrasound probe of claim 1, wherein the ultrasound transducer comprises one of a capacitive ultrasound transducer and a piezoelectric ultrasound transducer.

3. The ultrasound probe of claim 1, wherein the ultrasound transducer comprises:
at least one membrane; and
at least one cavity disposed between the surface of the first substrate and the at least one membranes.

4. The ultrasound probe of claim 1, wherein at least one of the first and second substrate comprise at least one of silicon, gallium arsenide, glass, and ceramic.

5. The ultrasound probe of claim 1, wherein the one or more non-imaging sensors are formed in the interior of the first substrate.

6. The ultrasound probe of claim 1, wherein the one or more non-imaging sensors are formed on the surface of the first substrate.

7. The ultrasound probe of claim 1, wherein the one or more non-imaging sensors are formed on the second substrate.

8. The ultrasound probe of claim 7, wherein the first and second substrates are connected by one or more vias.

9. The ultrasound probe of claim 1, wherein at least one of the non-imaging sensors are adjacent to the ultrasound transducer.

10. The ultrasound probe of claim 1, wherein at least one of the non-imaging sensors comprises a thermistor.

11. The ultrasound probe of claim 1, wherein at least one of the non-imaging sensors comprises a position sensor.

12. The ultrasound probe of claim 1, wherein at least one of the non-imaging sensors comprises an optical sensor.

13. The ultrasound probe of claim 12, wherein the optical sensor comprises a bar code reader.

14. The ultrasound probe of claim 1, wherein at least one of the non-imaging sensors comprises an electromagnetic sensor.

15. The ultrasound probe of claim 1, wherein at least one of the non-imaging sensors comprises a pressure sensor.

16. The ultrasound probe of claim 1, wherein at least one of the non-imaging sensors comprises a strain sensor.

17. The ultrasound probe of claim 1, wherein at least one of the non-imaging sensors comprises an ultrasound ranging sensor.

18. The ultrasound probe of claim 1, further comprising a lens over the ultrasound transducer.

19. The ultrasound probe of claim 1, further a backing underlying the ultrasound transducer and the one or more non-imaging sensors.

20. An ultrasound imaging system, comprising:
an ultrasound probe comprising:
an ultrasound transducer formed on a surface of a first substrate; and
one or more non-imaging sensors formed on the first substrate or on a second substrate proximate to the first substrate;
an acquisition module configured to acquire a set of imaging data from the ultrasound transducer and a set of non-imaging data from the non-imaging sensors;
a processing module configured to process at least the set of imaging data;
an operator interface configured to control the operation of at least one of the ultrasound probe, the acquisition module, and the processing module; and
a display unit coupled to the processing module, configured to display at least the processed imaging data.

21. The ultrasound imaging system as recited in claim 20, wherein the ultrasound probe is configured to vary its operation based upon the set of non-imaging data.

22. The ultrasound imaging system as recited in claim 20, wherein the acquisition module acquires the set of imaging data based upon the set of non-imaging data.

23. The ultrasound imaging system as recited in claim 20, wherein the processing module processes the set of imaging data based upon the set of non-imaging data.

24. A method for acquiring imaging and non-imaging data, comprising:
contacting an ultrasound probe to an imaging subject, wherein the ultrasound probe comprises an ultrasound transducer formed on a surface of a first substrate and one or more non-imaging sensors formed on the first substrate or on a second substrate proximate to the first substrate;
acquiring imaging data via the ultrasound transducer; and
acquiring non-imaging data via the one or more non-imaging sensors.

25. The method as recited in claim 24, wherein the non-imaging data comprises temperature data.

26. The method as recited in claim 25, comprising:
regulating an operational state of the ultrasound probe based on the thermal data.

27. The method as recited in claim 24, wherein the non-imaging data comprises at least one of position data, optical data, electromagnetic data, pressure data, and strain data.

28. The method as recited in claim 24, wherein acquiring imaging data comprises acquiring imaging data based upon the non-imaging data.

29. The method as recited in claim 24, comprising:
processing the imaging data based on the non-imaging data.

30. The method as recited in claim 29, wherein processing the imaging data comprises generating an ultrasound image.

31. The method as recited in claim 29, wherein processing the imaging data comprises relating the imaging data to a record or history of the imaging subject.

32. A method for constructing an ultrasound probe, comprising
forming an ultrasound transducer on a surface of a first substrate; and
forming one or more non-imaging sensors on the first substrate or on a second substrate proximate to the first substrate.

33. The method as recited in claim 32, wherein at least one of the first substrate and the second substrate comprise at least one of silicon, gallium arsenide, glass, and ceramic.

34. The method as recited in claim 32, wherein at least one of the first substrate and the second substrate are supported by an acoustically lossy material.

35. The method as recited in claim 32, wherein forming the ultrasound transducer comprises forming the ultrasound transducer by micro-electric mechanical systems techniques:

36. The method as recited in claim 32, wherein forming the ultrasound transducer comprises forming the ultrasound transducer by semiconductor processing techniques.

37. The method as recited in claim 32, wherein forming the one or more non-imaging sensors comprises forming the non-imaging sensors by micro-electric mechanical systems techniques.

38. The method as recited in claim 32, wherein forming the one or more non-imaging sensors comprises forming the one or more non-imaging sensors by semiconductor processing techniques.

39. The method as recited in claim 32, wherein forming the one or more non-imaging sensors comprises forming the one or more non-imaging sensors on an interior layer of the first substrate.

40. The method as recited in claim 32, wherein forming the one or more non-imaging sensors comprises forming the one or more non-imaging sensors on the surface of the first substrate.

41. The method as recited in claim 32, wherein forming the one or more non-imaging sensors comprises forming the one or more non-imaging sensors on the second substrate.

42. The method as recited in claim 41, the first and second substrates are connected by one or more vias.

43. The method as recited in claim 32, wherein the one or more non-imaging sensors comprise at least one of a thermistor, a position sensor, an optical sensor, an electromagnetic sensor, a pressure sensor, and a strain sensor.
